(19) United States
(12) Patent Application Publication
(10) Pub. No.: US 2017/0325840 A1
(43) Pub. Date: Nov. 16, 2017

(54) Percutaneous Gastrointestinal Access System and Method

(71) Applicant: Jason Benjamin Wynberg, Southfield, MI (US)

(72) Inventor: Jason Benjamin Wynberg, Southfield, MI (US)

(21) Appl. No.: 15/155,053

(22) Filed: May 15, 2016

(51) Int. Cl. A61B 17/34; A61B 17/00; A61B 1/018

(52) U.S. Cl. CPC A61B 17/3415; A61B 17/00234; A61B 17/3496; A61B 1/018; A61B 2017/00336; A61B 2017/0034; A61B 2017/00278

(57) ABSTRACT

A method for creating a tract for percutaneous endoscopic guided gastrointestinal tube creation including the steps of providing a puncture wire having a sharp tissue penetrating tip shielded in a sheath, the puncture wire slidable within the sheath and releasably lockingly engaged thereto; inserting the puncture wire and sheath in a first direction through a working channel of an endoscope to exit the channel of the endoscope, then releasing the puncture wire from the sheath and advancing the puncture wire from the sheath while visualizing via the endoscope the position of the puncture wire, and further advancing the puncture wire through the visceral wall and then the abdominal wall of a patient. Subsequent methods to position a percutaneous gastrointestinal catheter after initial wire puncture are described.

### BACKGROUND

This application claims priority from provisional application No. 62/162,808, filed May 17, 2015. The entire contents of this application are incorporated herein by reference.

### TECHNICAL FIELD

This application relates to a percutaneous puncture system and more particularly to a percutaneous puncture system and method for creating a tract for access to the gastrointestinal system.

### BACKGROUND OF RELATED ART

Gastrostomy is the creation of a communication between the skin and stomach to provide for percutaneous access to the stomach for provision of nutrition, fluids or other reasons. Similarly, jejunostomy is the creation of a communication between the skin and jejunum to provide for percutaneous access to the jejunum for provision of nutrition, fluids, or other reasons. As well, cecostomy is the creation of a communication between the skin and the cecum to provide access to the cecum e.g., to instill fluid for antegrade enemas.

Current percutaneous gastrointestinal tract creation (e.g. gastrostomy, jejunostomy, cecostomy) is most commonly performed either by open surgery, or using a minimally invasive technique (e.g., fluoroscopically guided, laparoscopic assisted or robotic-assisted).

There are two general techniques used for minimally invasive gastrointestinal tube placement. Both techniques share an initial step whereby a rigid needle cannula is initially advanced in the direction from outside the patient to inside the target lumen (e.g., stomach, jejunum, or cecum).

In the first minimally invasive technique, the catheter is deployed in an antegrade direction, from outside the skin to inside the abdomen/viscera lumen. This can be achieved using a Seldinger technique or a one-stick trocar method (e.g., COOK® Medical “Deutsch gastrostomy catheter set”). In many cases, suture anchor(s) (e.g., COOK® Medical “Chait suture anchor set—enterostomy configuration” or Boston ^{Scientific}® “T-Fastener”) are used to secure the viscera against the abdominal wall during tract dilation and wire/catheter deployment, thereby reducing the risk of injury to other structures (e.g., other bowel segments) and errant intraperitoneal placement of the catheter. In some cases, the initial catheter serves to mature the tract, after which a different catheter is placed for longer term use (e.g., Cook Medical Chait Trapdoor Cecostomy Catheter).

In the second minimally invasive technique, a guidewire with a loop on the distal end is advanced through the rigid needle cannula and snared by a grasper introduced through the gastroscope working channel (e.g., Boston Scientific® “EndoVive One-Step Button”). The gastroscope is removed from the mouth, thereby drawing the looped end of the guidewire out of the mouth. A string with a loop that was pre-attached to a gastrostomy tube is then attached to the looped end of the guidewire. Following this, the guidewire is gently pulled out the abdominal wall, thereby drawing the gastrostomy tube from its original position outside the mouth to the inside of the stomach and continuing until a portion of the gastrostomy tube is outside of the abdominal wall skin with the other end inside the stomach lumen.

In both of the foregoing techniques, precise targeting of the target viscera is difficult to achieve when the needle is advanced antegrade through the abdominal wall, and such antegrade approach can cause serious problems. The needle could miss the target puncture site and can injure blood vessels (e.g., gastroepiploic vessels), thereby causing hemorrhage or injure other organs, viscera, etc. In other instances, the needle cannula could pass completely through the opposite wall of the organ. Additionally, multiple stabs may be required to reach the target, thereby increasing the risk of infection, pain, etc.

The first technique suffers from further problems as multiple needle cannula passages are required to deploy suture anchor(s) and the suture anchors may injure the target viscera if excess traction is applied to them. As well, after the procedure the anchors need to pass through the gastrointestinal tract which can cause bleeding and/or distress. Insufflation and prior radioopaque contrast may need to be employed to improve puncture site targeting.

The second technique also suffers from additional deficiencies as it requires a separate snare to capture the looped wire, and successful snaring can be difficult. The technique also requires a separate snare device.

By performing a retrograde puncture of the visceral and abdominal walls rather than using the aforedescribed antegrade approach, the operator can create a more precisely located tract. This improved anatomic positioning is anticipated to reduce complications. The retrograde technique further allows ‘through and through’ control over the puncture wire by securing the puncture wire at or outside of the endoscope as well as outside of the skin at the puncture site. This enhanced control of the puncture wire, as compared to antegrade rigid needle cannula, may permit easier and more secure introduction of a drainage catheter, either single or coaxial lumen, over the puncture wire in an antegrade direction (opposite the direction of puncture) into the target viscera lumen by stabilizing the member over which the catheter or member is introduced.

I. S. Gill (U.S. Pat. No. 5,851,195) describes a method of retrograde puncture for jejunostomy creation. However, Gill technique has many deficiencies.

First, Gill discloses a wire in the form of a thin cannula or with an inner lumen. Such wire has inferior puncture ability as it precludes creation of a very sharp conical or parabolic point as can be created at the end of a solid wire. Second, Gill's method discloses a puncture member comprised of both the puncture cannula and the sheath together which increases the total diameter of the puncture member (wire and sheath together), potentially leading to failed punctures due to increased resistance from the larger total diameter. Third, Gill does not disclose a procedure for first delivering the wire out of the abdomen before securing the wire in relation to the sheath.

Additionally, Gill describes advancement of the puncture wire within a simple sheath without a method described to secure the two in relation to each other during advancement. The physician thus inserts the sheath and wire combination into the delicate working channel of the endoscope with these two components freely movable in relation to each other. This deficiency risks the puncture wire emerging from the sheath during passage through the endoscope, thereby causing damage to the lining of the endoscope working channel and failure of delivery of the puncture wire out of the end of the working channel. Additionally, advancement of the puncture wire through the sheath is performed by the surgeon holding the sheath in one hand while advancing the puncture member through the sheath with the other hand. The outer diameter of the sheath is dimensioned much less than the inner diameter of the endoscope working channel, and this provides a small surface area of sheath to pinch while controlling the sheath's position, likely causing compression on the puncture wire that is inside the sheath, thereby restricting advancement of that wire.

Finally, Gill specifies that the puncture wire is preferably a maximum of 20 cm longer than the protective sheath. This is inadequately short and does not provide sufficient distance of puncture wire beyond the sheath to permit subsequent procedural steps in some applications.

Consequently, the need exists for an improved retrograde approach for creating a tract to access the gastrointestinal system. It would be advantageous if such approach improved puncturing effect of the puncture wire, provided more control of the puncture wire and protective sheath during advancement, facilitated advancement of the puncture wire and minimized the diameter of the components. The need exists for such improved approach which would also minimize the risks to the patient of failed punctures or the risk of failed proper advancement of the puncture wire.

### SUMMARY

The present invention overcomes the disadvantages and deficiencies of the prior art. The present invention provides in one aspect a method for creating a tract in retrograde fashion for gastrointestinal tract creation comprising the steps of providing a puncture wire having a tissue penetrating tip shielded in a sheath and inserting the puncture wire and sheath through a channel in an endoscope. The method further includes the steps of advancing the puncture wire from the sheath while visualizing under direct vision the position of the puncture wire and advancing the puncture wire from inside the lumen of the target organ (e.g. stomach, jejunum, or cecum) to outside the skin approximately overlying that target organ.

In some embodiments, the sheath and puncture wire are releasably locked together such as with a pin vise lock that in some embodiments is permanently mounted on the proximal end of the sheath, and the method can further comprise the step of releasing the puncture wire from the sheath.

In some embodiments, the pin vise lock that is mounted at the back of the sheath serves two purposes during the procedure: first, to secure the puncture wire tip inside the sheath during advancement into the endoscope thereby preventing damage to the endoscope working channel; second, to re-lock the puncture wire to the sheath after the puncture wire has punctured the viscera and emerged from the abdominal wall.

In some embodiments, the wire and/or sheath are releasably fixable to the endoscope.

In some embodiments, the sheath is constructed of transparent or a translucent material, permitting visualization of a second member passed inside the lumen to determine a position of the second member in relation to relevant structures.

In some embodiments the puncture wire is composed of solid material, with a sharp tapered point on the distal end which can be conical shaped.

In some embodiments, the puncture wire may have a larger diameter at a proximal region and a narrower diameter at a distal region. This may be achieved with any number of ways, e.g., precision grinding to narrow the distal segment. Distal to the distal segment a sharp point may be ground, thus creating three segments—a larger proximal segment, a ground middle segment, and a sharp point distal to the middle segment. The larger diameter proximal segment may provide greater stiffness or kink resistance during advancement of a second catheter in a direction opposite to initial puncture or may alternatively provide a zero clearance to the puncture wire sheath that is sized to subsequently receive a larger second member internally (e.g., looped guidewire) and thereby optimize delivery out of the abdominal wall while still permitting a narrower puncture segment for puncture.

The method of use can include the step of advancing the puncture wire from inside the lumen of the target organ to emerge from the skin approximately overlying that target organ, with subsequent control of the puncture wire with a hemostat or similar clamp. The method can also include the step of then re-locking the pin vise lock after the puncture wire has emerged from the skin, thereby securing the puncture wire in relation to the sheath after the puncture wire has emerged at the skin.

The method can in some embodiments include the step of gently advancing the sheath in the same direction as the puncture wire, possibly with application of gentle traction on the puncture wire outside of the skin, also in the same direction as the puncture, until the sheath emerges from the skin and is controlled.

The method can in some embodiments include the step of removing the puncture wire from the sheath and advancing another member inside the sheath or overtop of the sheath. In some embodiments, this member is a guidewire. In some embodiments, this guidewire has an apparatus for fixation (e.g., a loop) on one end. This second member may pass in a direction the same as or opposite to the direction of initial wire puncture.

In one embodiment, this member is advanced opposite the direction of puncture, through the sheath from outside the skin to inside the patient's body, after which the endoscope and sheath are removed, leaving the second member ‘through and through’ from outside the abdominal skin to outside either the mouth of the patient (e.g., gastrostomy or jejunostomy) or anus of the patient (e.g., cecostomy). In another embodiment, this member is advanced in the same direction of puncture, through the sheath from inside the patient's body to outside the skin, after which the endoscope and sheath are removed, leaving the second member ‘through and through’ from outside the abdominal skin to outside either the mouth of the patient (gastrostomy or jejunostomy) or anus of the patient (cecostomy).

In some embodiments, a member (e.g., a needle cannula or single or coaxial lumen catheter) may be passed overtop of this ‘through and through’ controlled puncture wire, providing the puncture wire with a first function of tract puncture and a second function of providing luminal support for advancement of the member in second direction, opposite the direction of puncture. Safe antegrade advancement of a second member through a non-established tract is facilitated by distension of the viscera, through-and-through traction on the puncture wire to provide good luminal support, and a tapered second member that provides smooth clearance between the tip of the second member and the puncture wire.

In another aspect, the present invention provides a method for creating a tract for gastrointestinal tube creation comprising the steps of:
a) providing a puncture wire having a sharp tissue penetrating tip for penetrating an abdominal wall;
b) providing a sheath with a releasably locking lock mounted on its proximal end, the puncture wire slidable within the sheath and being selectively releasably lockable with the sheath;
c) with the penetrating tip shielded within the sheath and the lock in the locked position to prevent damage to a working channel of an endoscope from an exposed penetrating tip, advancing the puncture wire and sheath together into and through an end of the working channel of the endoscope positioned in a lumen of a target viscera;
d) releasing the lock;
e) advancing the puncture wire in relation to the sheath through the selected wall of the target viscera and the abdominal wall until the puncture wire emerges at a skin of a patient;
f) locking the lock to secure a position of the puncture wire in relation to the sheath;
g) advancing the puncture wire and sheath further out of the abdomen; and
h) removing the puncture wire from the sheath while leaving the sheath in position extending outside the abdominal wall to provide a through sheath to subsequently receive a member therethrough.

The method in some embodiments includes introducing the endoscope through a natural body orifice (for example: mouth for stomach or jejunum; anus for cecum), and establishing safe tract location with aid of light transillumination through skin. In some embodiments, the lock is a vise lock. In some embodiments, the member is a looped guidewire.

In another aspect, the present invention provides a method for creating a tract in retrograde fashion for percutaneous access to the gastrointestinal tract comprising the steps of:
a) providing a puncture wire having a sharp tissue penetrating tip for penetrating an abdominal wall;
b) providing a sheath with a releasably locking lock on its proximal end, the puncture wire slidable within the sheath and being selectively releasably lockable with the sheath;
c) with the penetrating tip shielded inside the end of the sheath and the lock in the locked position to prevent damage to a working channel of an endoscope from an exposed penetrating tip, advancing the puncture wire and sheath together into and through an end of a working channel in an endoscope that is positioned in the lumen of the target viscera;
d) releasing the lock;
e) advancing the puncture wire in relation to the sheath, through a selected visceral wall until the puncture wire passes through the abdominal wall and emerges at a skin of a patient.
f) drawing an additional length of the puncture wire out of the skin;
g) locking (e.g., closing) the lock to secure the puncture wire in position in relation to the sheath;
h) loading a member over the puncture wire at the skin and advancing the member through the abdominal wall and visceral wall into a viscera lumen; and
i) removing the puncture wire and securing the member in position, a portion of the member extending out of the abdominal wall.

In some embodiments, the method includes introducing the endoscope through a natural body orifice (for example: mouth for stomach or jejunum; anus for cecum), and establishing safe tract location with aid of light transillumination through skin. In some embodiments, the puncture wire has a fixation loop at the proximal end. In some embodiments, the lock is a vise lock mounted permanently on the proximal end of the sheath. In some embodiments, a single or coaxial lumen catheter is loaded over the puncture wire at the skin and advanced antegrade (opposite direction of retrograde wire puncture) until it is positioned inside the visceral lumen. The catheter can be fixed to the skin

In some embodiments the puncture wire may have a fixation apparatus (e.g., a loop) at the end opposite the puncture point (penetrating tip). This loop is employed to fix another member (e.g., a gastrostomy, cecostomy, or jejunostomy drainage catheter) for delivery into position using the “pull” technique.

In another aspect, the present invention provides a method for creating a tract for gastrointestinal tube creation comprising the steps of:
a) providing a puncture wire having a sharp tissue penetrating tip at an end;
b) providing a sheath with a releasably locking vise lock mounted permanently on its proximal end, the puncture wire slidable within the sheath and being selectively releasably lockable with the sheath;
c) with the penetrating tip inside the end of the sheath and the lock in the locked position to prevent movement of the puncture wire and sheath in relation to each other and thereby avoid emergence of the puncture wire tip from the sheath, thus avoiding damage to a working channel of an endoscope from an exposed puncture wire tip, advancing the puncture wire and sheath together into and through the end of the working channel in the endoscope that is positioned in a lumen of a target viscera;
d) releasing the lock:
e) advancing the puncture wire in relation to the sheath, through a selected visceral wall until the puncture wire emerges through the abdominal wall and is controlled at a skin of a patient;
f) removing the endoscope and sheath from the patient in a direction opposite a direction of insertion of the endoscope;
g) connecting a gastrointestinal catheter to the proximal end of the puncture wire located at a natural body orifice;
h) drawing the end of the puncture wire further out of the abdominal wall to draw the gastrointestinal catheter into the target viscera, and
i) removing of the puncture wire, leaving the gastrointestinal catheter in proper position.

In some embodiments, the method includes introducing the endoscope through a natural body orifice (for example: mouth for stomach or jejunum; anus for cecum), and establishing safe tract location with aid of light transillumination through skin. In some embodiments, the wire is gently secured at the skin.

It is contemplated that for all three systems described herein, an endoscopic, laparoscopic or robotic assisted procedure may be used to assist in delivery of the puncture wire from inside the abdominal cavity to outside the abdominal wall, by advancing a grasping or directing member positioned inside the abdominal cavity to grasp the puncture wire after it penetrates out of the viscera. By doing so, the puncture wire that has emerged from inside to outside the viscera is delivered out of the abdomen with a surgeon-assisted minimally invasive (endoscopic, laparoscopic or robotic) technique.

In some embodiments the puncture wire is a solid construction. In some embodiments, the puncture wire has a fixation loop at the proximal end which is connectable to a loop on a catheter. In some embodiments, the lock is a vise lock. The vise lock can be mounted permanently on the proximal end of the sheath;

The present invention in another aspect provides a kit for creating a tract in retrograde fashion for percutaneous access to the gastrointestinal tract comprising a) a puncture wire having a first diameter for insertion through an abdominal wall and (b) a sheath having a length, the puncture wire slidable with respect to the sheath, and the sheath removable from the puncture wire.

### BRIEF DESCRIPTION OF THE DRAWINGS

Preferred embodiment(s) of the present disclosure are described herein with reference to the drawings wherein:

FIG. 1 is a side view of the puncture wire and sheath with mounted pin vise lock of the present invention;

FIG. 2 is a side view of the puncture wire of FIG. 1 extending beyond the end of the sheath, thereby exposing the puncture wire tip;

FIG. 3 is a perspective view of pin vise system of FIG. 1;

FIG. 3b is a side view of the pin vise apparatus;

FIG. 4 is a side view illustrating the endoscope inserted into the stomach or jejunum via the patient's mouth;

FIG. 5 is a top down perspective view of the endoscope inserted into the cecum via the patient's anus;

FIG. 6 is a side view of the puncture wire with a narrower distal segment and puncture point distal to the narrow distal segment in accordance with one embodiment of the present invention;

FIG. 7 is a side view of a solid core puncture wire with loop on proximal end and puncture point on a distal end in accordance with another embodiment of the present invention.

FIG. 8 is a side view of a guidewire with a looped segment on a proximal end and a non-sharp tip on a distal end;

FIG. 9 is a schematic view of the endoscope inside the visceral lumen with the puncture wire advanced out of the sheath and through abdominal wall skin;

FIG. 10 is a side view of the puncture/sheath apparatus inserted into the working channel of the endoscope, with the pin vise lock being re-locked to secure the puncture wire in relation to the sheath;

FIG. 11 is a perspective view showing the sheath releasably secured to the endoscope with a Touhy-Borst device mounted on the endoscope;

FIG. 12 is cut away view of Touhy Borst device of FIG. 11 compressing a circular O-ring against the sheath;

FIG. 13 is a view similar to FIG. 9 illustrating the sheath further extended through the skin and the sheath advanced over the puncture wire through the abdominal wall;

FIG. 14 is a view similar to FIG. 13 illustrating the looped end of a second member guidewire being advanced into the end of the sheath exposed at the skin;

FIG. 15 is a view of the looped end of a second member guidewire emerging at the mouth;

FIG. 15a is a view of the looped end of a second member guidewire emerging at the anus;

FIG. 16 is view of a gastrointestinal tube with looped segment connected to the loop on guidewire at the mouth end of patient;

FIG. 17 is a view of a gastrointestinal tube of FIG. 16 in position, bridging the visceral and abdominal walls;

FIG. 18 is a view illustrating advancement of a catheter over the puncture wire and into the patient;

FIG. 19 is a view of the catheter of FIG. 18 in position, bridging the visceral and abdominal walls;

FIG. 20 is a side view after removal of the endoscope, showing the distal end of the puncture wire outside of the abdomen and the proximal, looped end outside of the mouth; and

FIG. 21 is a top perspective view kit depicting the puncture wire, sheath with pin vise lock, and an optional catheter in accordance with one embodiment of the present invention.

### DETAILED DESCRIPTION OF PREFERRED EMBODIMENTS

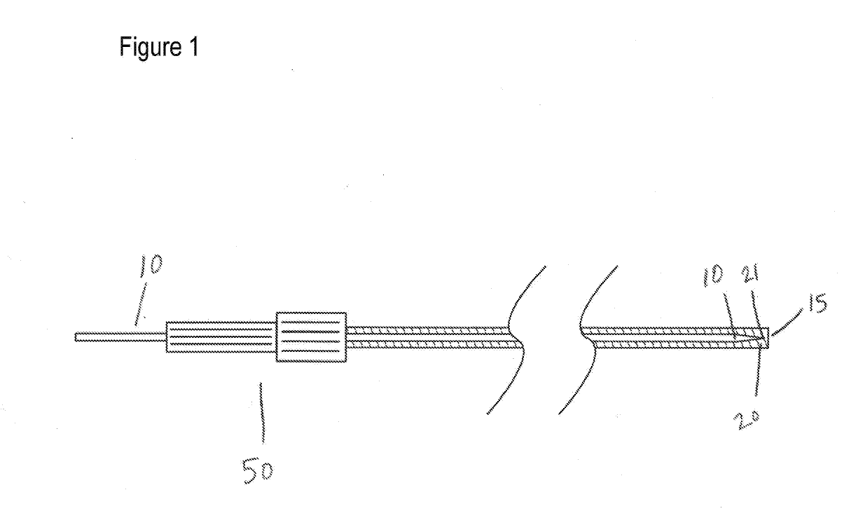

Referring now in detail to the drawings wherein like reference numerals identify similar or like components throughout the several views, FIGS. 1-21 illustrate various embodiments of the present invention. The present invention provides percutaneous access to the gastrointestinal system, with various ways to position a catheter subsequent to puncture.

A puncture wire is advanced through a working channel of a gastrointestinal endoscope which has been passed into the gastrointestinal system in a retrograde fashion through the mouth or anus to the target viscera puncture site. This technique obviates the need for antegrade access to the target viscera as antegrade access disadvantageously requires a less precise, estimated puncture direction into target the viscera with the risk of missing the target viscera, causing bleeding, possibly requiring use of extra devices to secure the access e.g., snare device, suture anchors, etc. The retrograde approach of the present invention, as will become apparent from the detailed description below, not only provides improved visualization, but provides such improved visualization while securing/locking the positions of the sheath and wire with respect to each other and the endoscope, and ensuring the wire is protected from damaging the endoscope and tissue during the procedure. Further, the present invention, as also discussed below, enables a streamlined approach to placement of a percutaneous catheter into the gastrointestinal system.

Three systems and methods are provided by the present invention, all utilizing a puncture wire and a protective sheath with a releasably locking vise lock mounted on the proximal end of the sheath, or other locking feature to selective secure the puncture wire and sheath together.

In the first system and method described herein, the sheath which protects the puncture wire during insertion is also used as an exchange sheath so that the puncture wire can be withdrawn and a second member inserted into the sheath. In some embodiments, that second member is a looped guidewire which is advanced loop first into the sheath so that the loop emerges at the mouth. The endoscope and sheath are removed, leaving this wire guide ‘through and through’ the patient. A looped segment on the end of a gastrointestinal catheter can be attached to the looped end of the guidewire, and the guidewire can be drawn further out the abdominal wall until the catheter is in position. This simplifies the components and procedural steps of the surgery.

In the second system and method described herein, the puncture wire serves a dual function—first puncturing through the visceral wall and abdominal wall, and second as a support over which a second member with a lumen, for example a cannula or catheter, either single or coaxial design, can be loaded and advanced from outside the abdomen to inside the viscera.

In the third system and method described herein, a solid construction puncture wire has a loop on the proximal end so that after initial puncture through the visceral wall and then abdominal wall, the endoscope and sheath are removed and the loop at the proximal end can be secured to a looped segment on a gastrointestinal catheter to be drawn into position by drawing the puncture wire further out of the abdominal wall.

It is contemplated that for all three systems described herein, an endoscopic, laparoscopic or robotic assisted procedure may be used to assist in delivery of the puncture wire from inside the abdominal cavity to outside the abdominal wall, by advancing a grasping or directing member positioned inside the abdominal cavity to grasp the puncture wire after it penetrates out of the viscera. By doing so, the puncture wire that has emerged from inside to outside the viscera is delivered out of the abdomen with a surgeon-assisted minimally invasive (endoscopic, laparoscopic or robotic) technique.

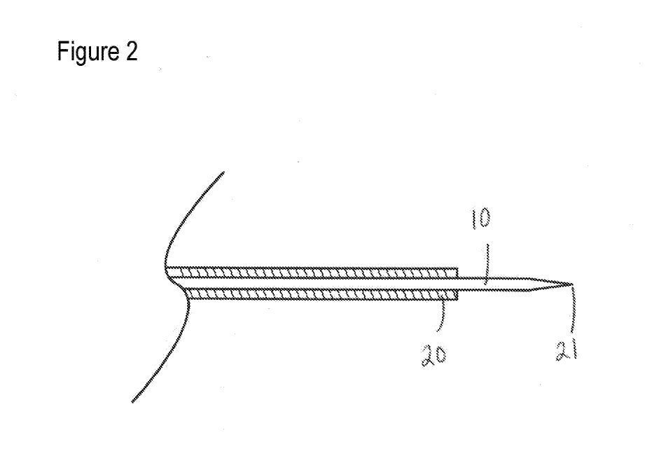

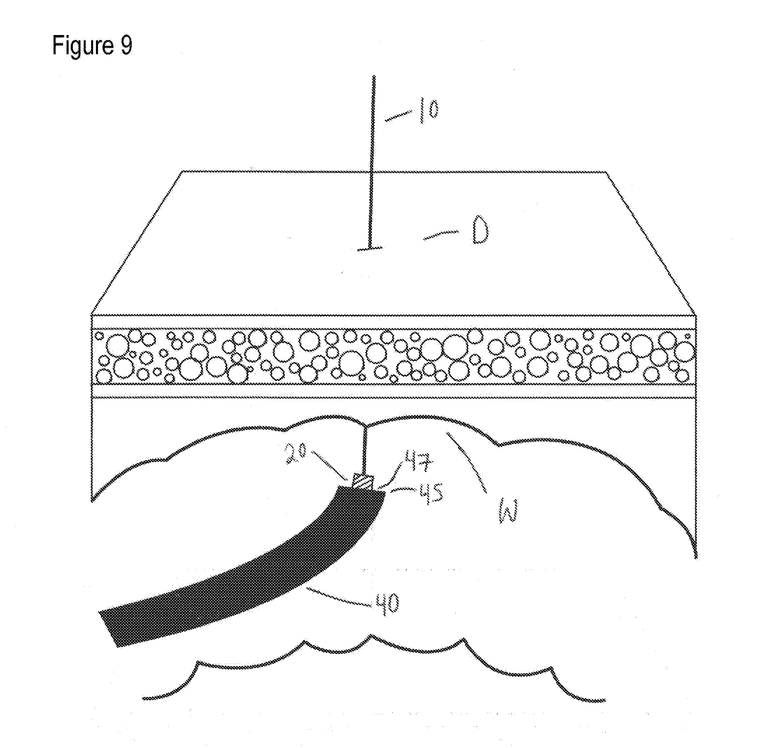

Turning initially to the first system and method which is illustrated in FIGS. 1-17, the system includes a protective sheath 20 and a puncture wire 10. Sheath 20 has a lumen 15 extending therethrough dimensioned to slidingly receive the puncture wire 10 therein. That is, puncture wire 10 is received within the sheath lumen 15 for sliding movement from a retracted position wherein the puncture (penetrating) tip 21 of wire 10 is protected (shielded) by the sheath 20 and an extended position (FIG. 2) where the puncture tip 21 is exposed from the sheath 20 to penetrate tissue (see e.g. FIG. 9) as the puncture tip 21 extends beyond the distal opening 24 of the sheath 20 (FIG. 2). Exposure of the puncture wire tip 21 enables advancement of the puncture wire 10 through the visceral wall and abdominal wall skin as described below.

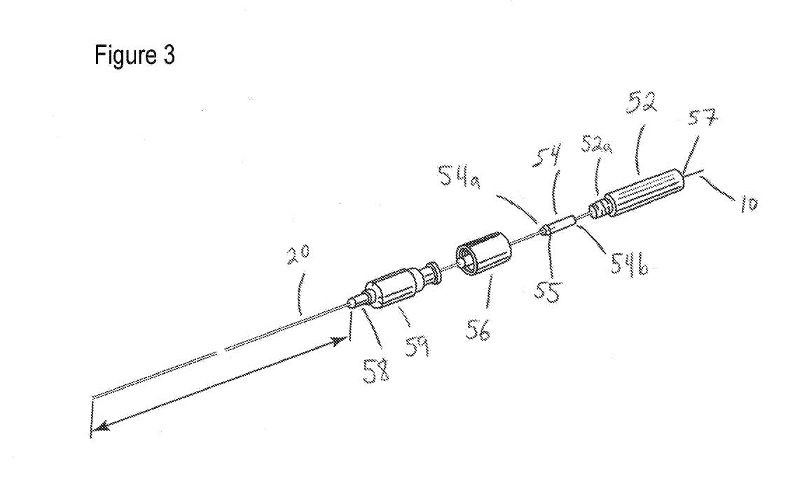

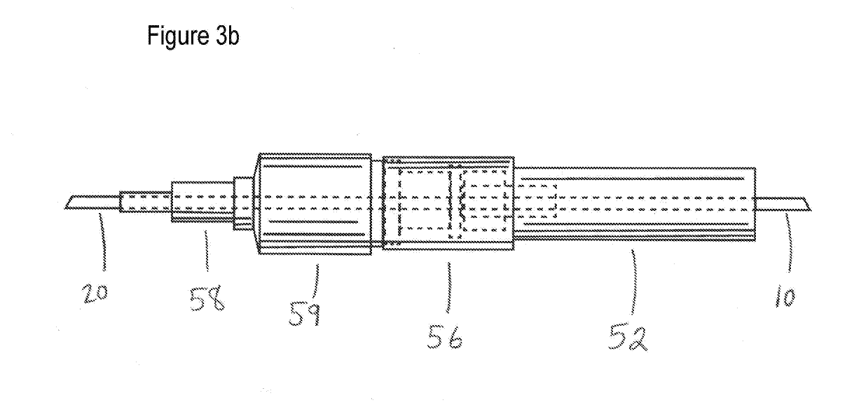

The puncture wire 10 and sheath 20 are releasably locked together by a conventional vise lock 50. Other locks to secure the puncture wire 10 and sheath 20 together are also contemplated. As shown, with reference to FIG. 1, 3, 3b, 10, vise lock 50 has a rotatable actuator 52 (FIG. 3) and a metal locking tube 54 with a longitudinally extending elongated slot 55. A first (distal) portion 54a of locking tube 54 is seated within tube 56; a second opposite proximal portion 54b is seated within the actuator 52 (FIG. 3). Actuator 52 has reduced diameter portion 52a threadingly received in tube 56 and a lumen 57 through which the wire 10 extends. When actuator 52 is rotated within tube 56, it clamps down on the metal locking tube 54 reducing its diameter due to the slot 55, to thereby clamp down on the wire 10 to lock it from sliding movement with respect to the sheath 20. Consequently, as described below with respect to the method of use, the wire 10 and sheath 20 can be locked together so they can be advanced as a unit through the gastrointestinal endoscope. When it is desired to move the puncture wire 10 relative to the sheath 20, the actuator 52 is unscrewed from tube 56, thereby releasing the clamping force on the metal tube 54 so the wire 10 can slide relative to the sheath 20. A reinforcement tube 58 extends distally from distal tube 59 which can connect via screw threads (or by other methods).

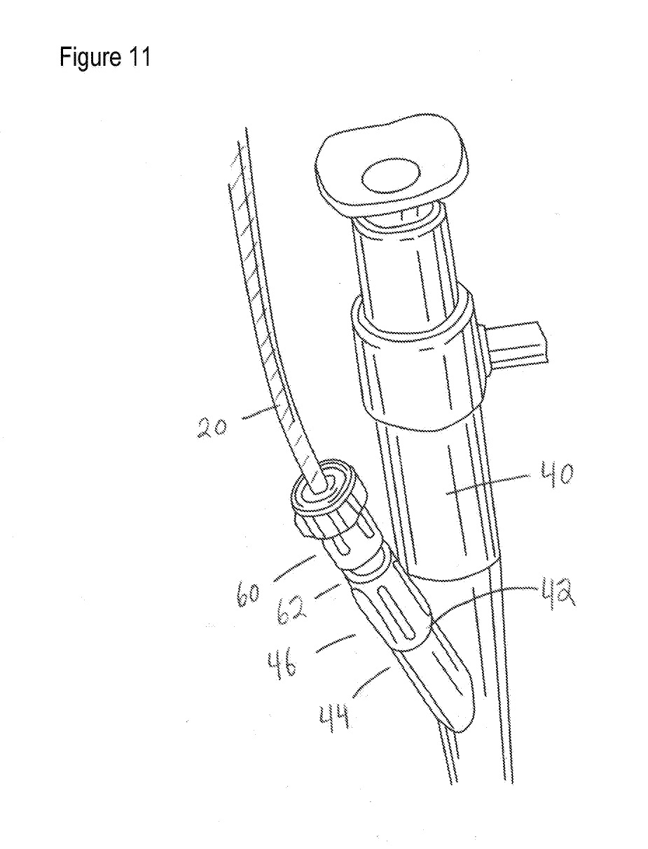

The region of the sheath 20 adjacent the vise lock can include a strengthened region to help stabilize the system. The strengthening can be achieved by thickening, reinforcing or hardening the sheath in this region (see e.g. reinforcement tube 58 of FIG. 3). This would reduce the movement of the pin vise during puncture wire deployment. FIG. 3b depicts a side vide of the pin vise lock. The system also includes a sheath locking mechanism 60 (FIG. 11) for locking the sheath 20 to a working channel of the endoscope 40. This is described in more detail below.

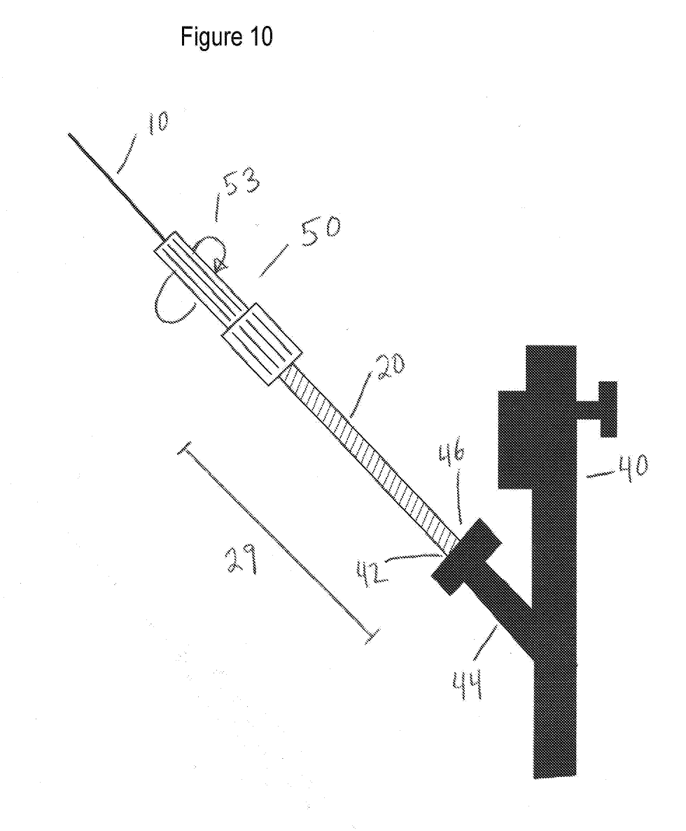

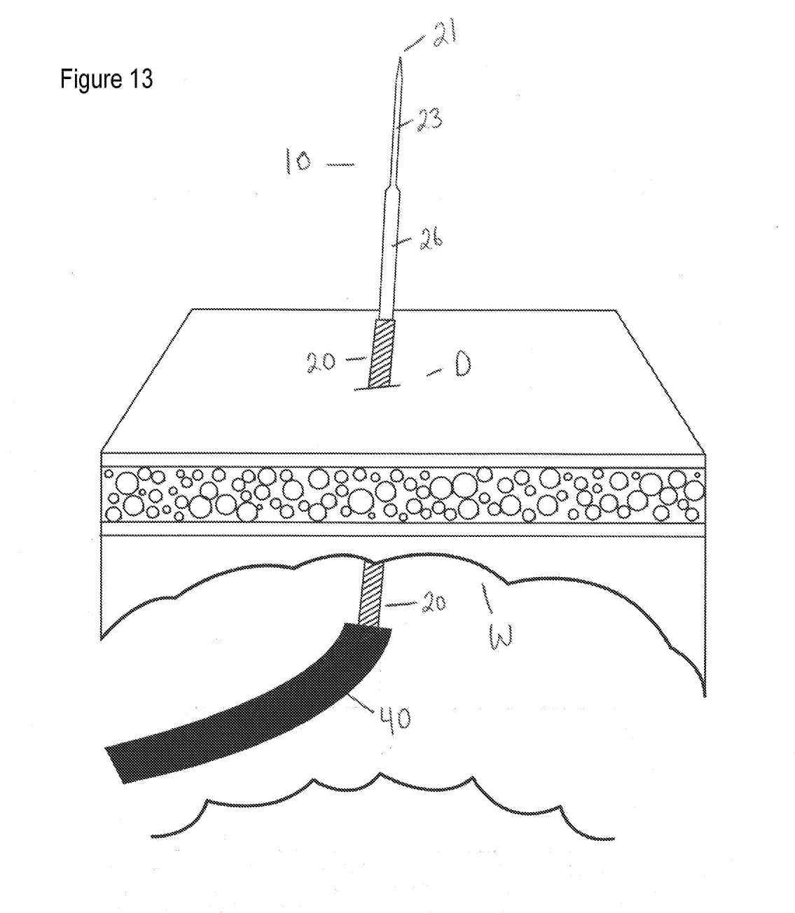

A conventional endoscope is designated generally by reference numeral 40 in Figures and includes a working (operating) channel opening 46 communicating with channel (lumen) 42 (FIG. 10). The endoscope 40 is preferably a steerable scope so it can be articulated through the gastrointestinal system to gain access to the desired site of tract puncture. The working channel 42 is accessible through an opening in side arm or port 44 which communicates with the endoscope channel 42 extending longitudinally within the length of the endoscope 40. The endoscope 40 provides both illumination and visualization of the surgical site as well as illumination and visualization of the puncture wire 10 and sheath 20 as they are advanced from the distal opening 47 of the endoscope 40 (e.g., FIGS. 9 and 13), thus providing viewing of the system components as well as the patient's body.

Note the port of the scope 40 prevents irrigation fluid leakage from the working (operating) channel, and can include a Tuohy-Borst type adapter which seals around instrumentation (e.g. the sheath) inserted therethrough. It can also tighten around the protective sheath with a circumferentially tightening O-ring mechanism.

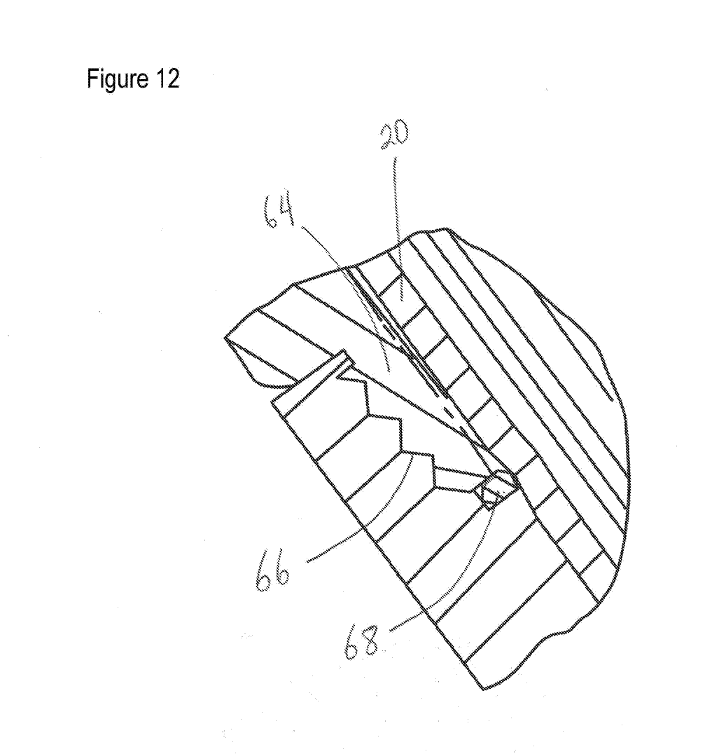

Sheath locking mechanism 60, as shown in FIG. 11, is mounted to side port 44 of endoscope 40. With reference to FIGS. 11 and 12, sheath locking mechanism 60 includes a scope mounting portion 62 having a tubular extension at its distal portion for insertion into the working channel 42 of the endoscope 40. The distal portion can have a threaded end for threading into the opening 46 of the working channel 42 or alternatively can be snap fit into opening 46 to attach the mounting portion 62 to the endoscope 40. A lumen extends though the mounting portion 62 and contains a flexible O-ring 68 positioned therein. The sheath 20 extends though the lumen and through the opening in the O-ring 68. When threaded locking knob 64 is rotated, it provides a clamping force on the threaded cylinder 66 which clamps down on the O-ring 68 to reduce the size of its opening, thereby clamping down on the sheath 20 to lock it into position, i.e., lock it against movement with respect to the endoscope 40. Note the clamping force is sufficient to secure the sheath 20, but limited to not lock the wire 10 positioned therein, thereby still enabling sliding movement of the wire 10 with respect to the sheath 20. This locking of the sheath 20 to the endoscope 40 advantageously enables the sheath position to be maintained while the surgeon manipulates the puncture wire 10, and the wire 10 can be separately secured to the apparatus by closing the vise lock 50.

It should be appreciated that the sheath locking mechanism 60 can be provided on the sheath 20 as packaged, or alternatively provided as a separate component. If provided as a separate component, it can optionally be packaged with the sheath 20 in a kit.

It should be appreciated that other mechanisms for locking the sheath 20 to the endoscope 40 are also contemplated which would retain the sheath position during procedure. For example, the puncture wire/sheath duo could mate and lock directly onto the endoscope working channel port either by prior removal of the nipple and directly locking the Luer lock end of the pin-vise apparatus to the working channel, or by locking the pin-vise apparatus onto a separate device that interfaces with the working channel port and puncture wire/sheath duo.

Note the portion of the protective sheath 20 nearest the pin vise lock 50 may be made stiffer so that when locked in position by clamp 64, there would be less motion of the pin vise mechanism during deployment of the puncture wire 10 by the surgeon.

The sheath 20 preferably has a length of between about 150 cm to about 250 cm although other lengths are contemplated. With this length, the sheath 20 has sufficient length for insertion through the entire working channel 42 of the endoscope 40 including the portion of channel within the endoscope handle, as well as sufficient length to exit therefrom and extend through the viscera and abdominal wall skin. The sheath is preferably a 2.5 to 5 French sheath, having an internal diameter that is sufficient to receive both the puncture wire 10, and a subsequent approximately 0.025 to 0.045 inch guidewire through the lumen 15. Other dimensions are also contemplated. The sheath is preferably composed of PTFE although other materials are also contemplated.

The puncture wire 10 preferably has a length of between about 150 cm to about 260 cm, although other lengths are contemplated. The wire 30 preferably has a diameter ranging from about 0.015 inches to about 0.050 inches, sized to enable sliding movement within sheath lumen 15. With this length, the puncture wire 10 has sufficient length for insertion through the entire working channel 42 of the endoscope 40 as well as sufficient length to exit therefrom and extend through the visceral wall and abdominal wall skin. The puncture wire can be composed of stainless steel, although other materials including but not limited to other materials, alloys, nitinol, or nitinol alloys. Other wire compositions are also contemplated. Note that other wire lengths are also contemplated.

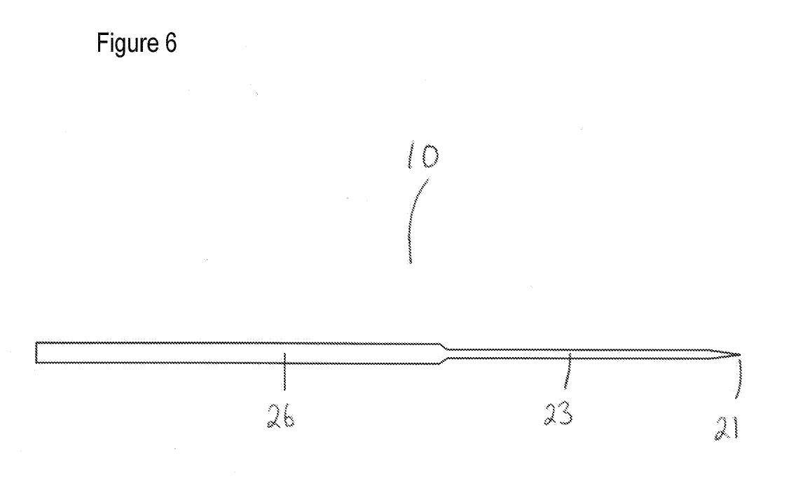

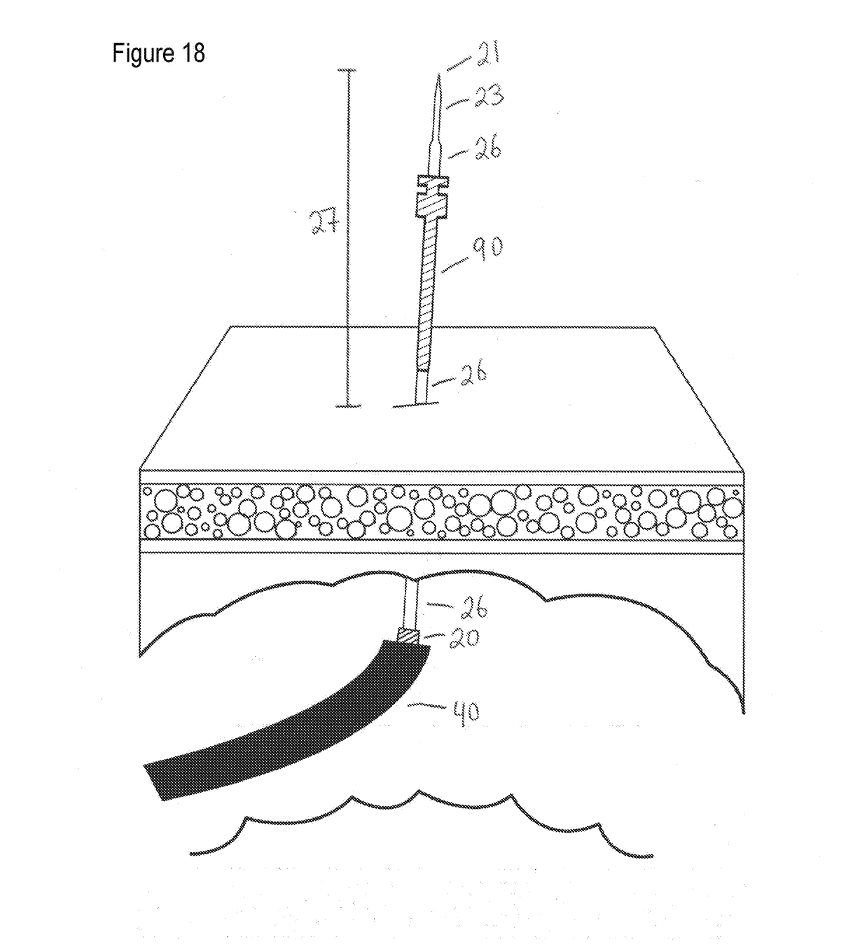

The puncture wire may have variable properties where each property is respectively optimized for various wire functions. In FIG. 6 one embodiment of a puncture wire 10 is illustrated. At the distalmost end of the wire is a sharp puncture point 21. Immediately proximal to the puncture point is a narrower segment of wire 23 whose diameter is wide enough to provide enough stiffness for puncture, yet enough flexibility to permit the distal end of the endoscope 40 to be deflected into position for puncture. A wire segment 26 proximal to the segment 23 is of larger diameter than segment 23 and this larger diameter proximal segment 26 is optimized to provide kink resistance during advancement of catheter 90 (FIG. 18) and to provide zero clearance to the sheath 20 during advancement of the sheath 20 from the visceral wall W and abdominal wall skin D as shown in FIG. 13. Narrower distal segment 23 may have a diameter between 0.014 inches and about 0.040 inches. Wider proximal wire segment 26 may have a diameter between 0.020 inches and about 0.050 inches. Other dimensions are also contemplated. Any number of methods are contemplated to create this variable property wire, including precision grinding to narrow a larger wire mandrel distally, addition of an investing sheath over top of a narrow mandrel to create the larger proximal segment 26, etc.

The puncture wire 10 in some embodiments has one or more markings on its outer surface to indicate to the surgeon its position with respect to the sheath 20, skin, and/or endoscope 40. The markings can be placed on a region of the puncture wire 10 extending outside the body or alternatively or additionally on a region extending within the body to be imaged by the endoscope 40. Likewise, the sheath 20 can have one or more markings on a region outside the body, e.g., adjacent sheath locking mechanism 60, or adjacent the pin-vise lock, and/or inside the body where the marking(s) can be visualized by the endoscope 40.

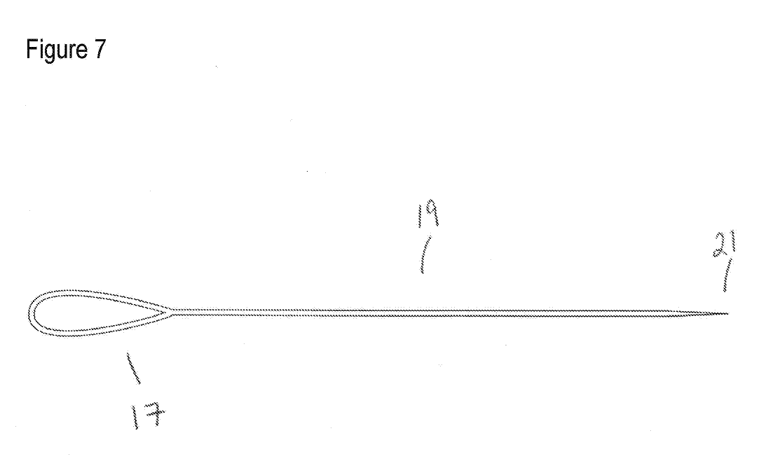

In another embodiment illustrated in FIG. 7, a solid core puncture wire 19 is provided with a looped proximal end 17. The looped segment 17 can be attached by bonding, as an integral extension of the body of the puncture wire or other methods to connect the looped segment to the body of the puncture wire 19. A sharp puncture point 21 is at the distal end of 19 variable property puncture wire 19. Note that such loop can be provided on any of the puncture wire embodiments disclosed herein.

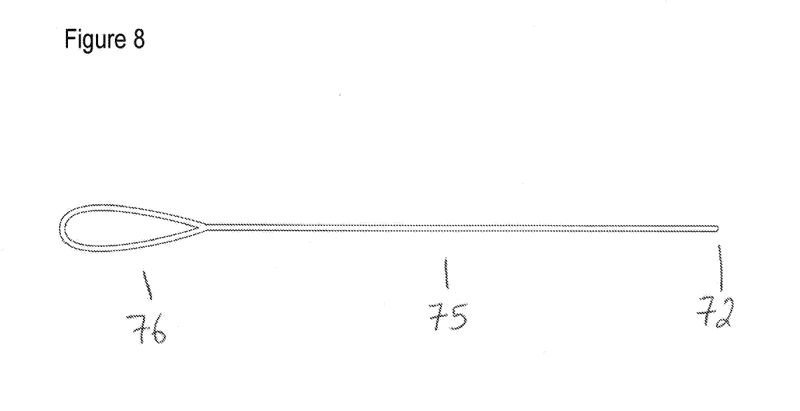

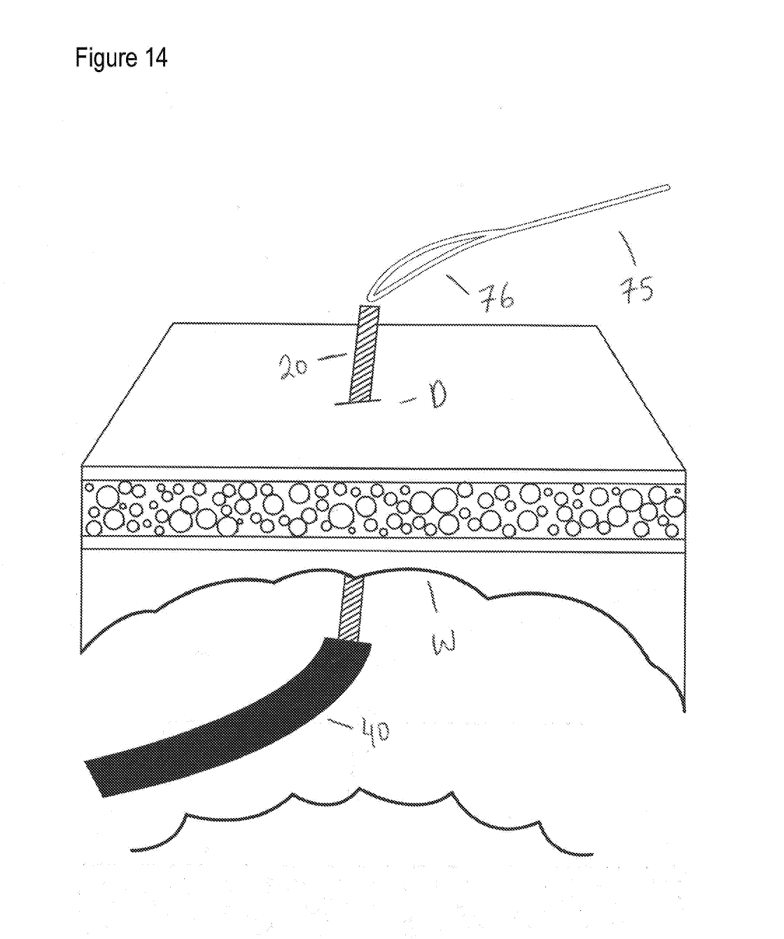

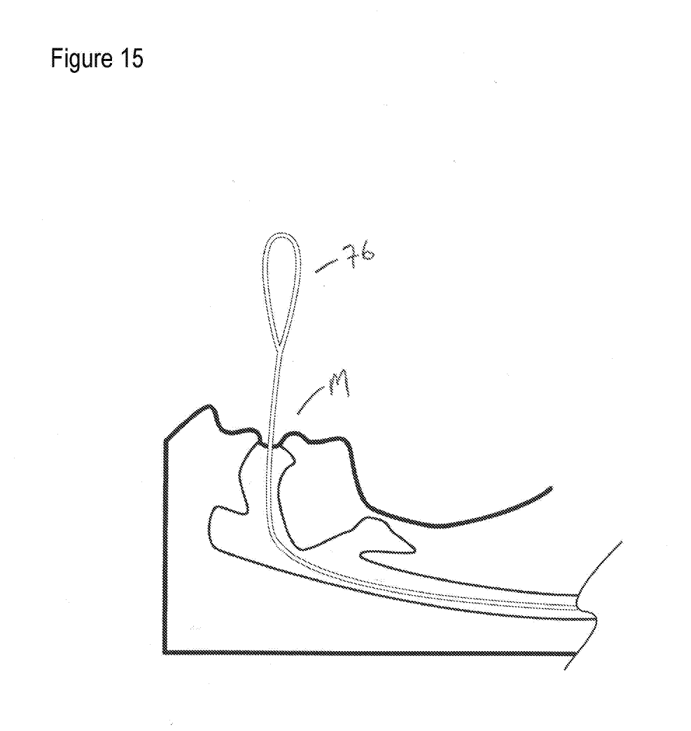

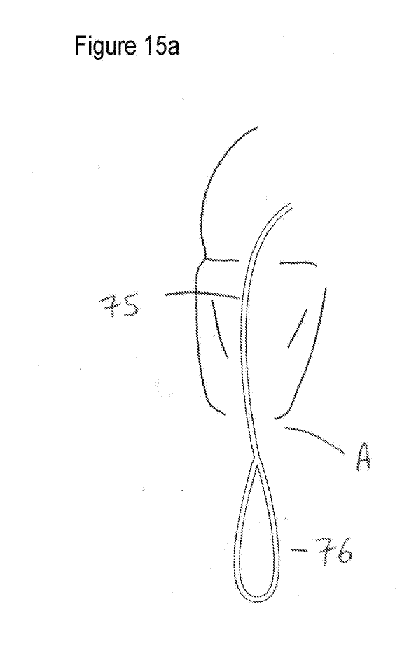

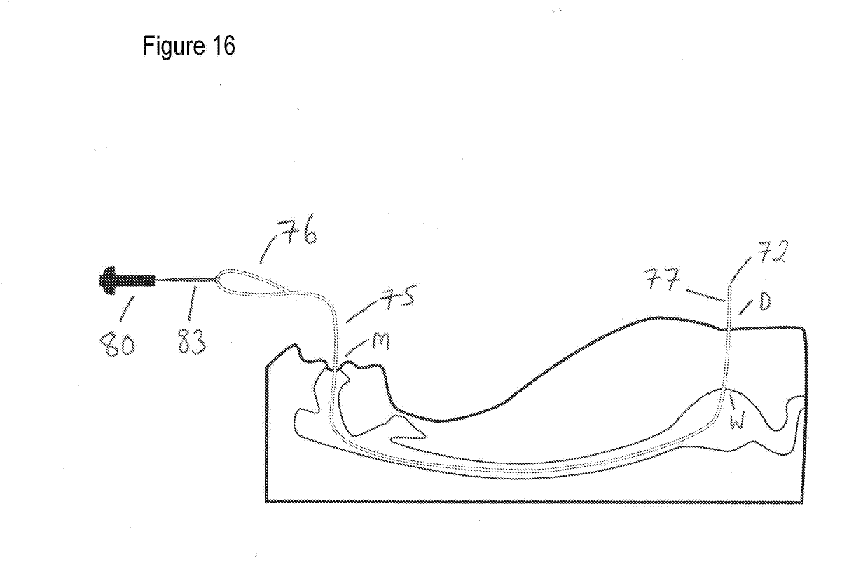

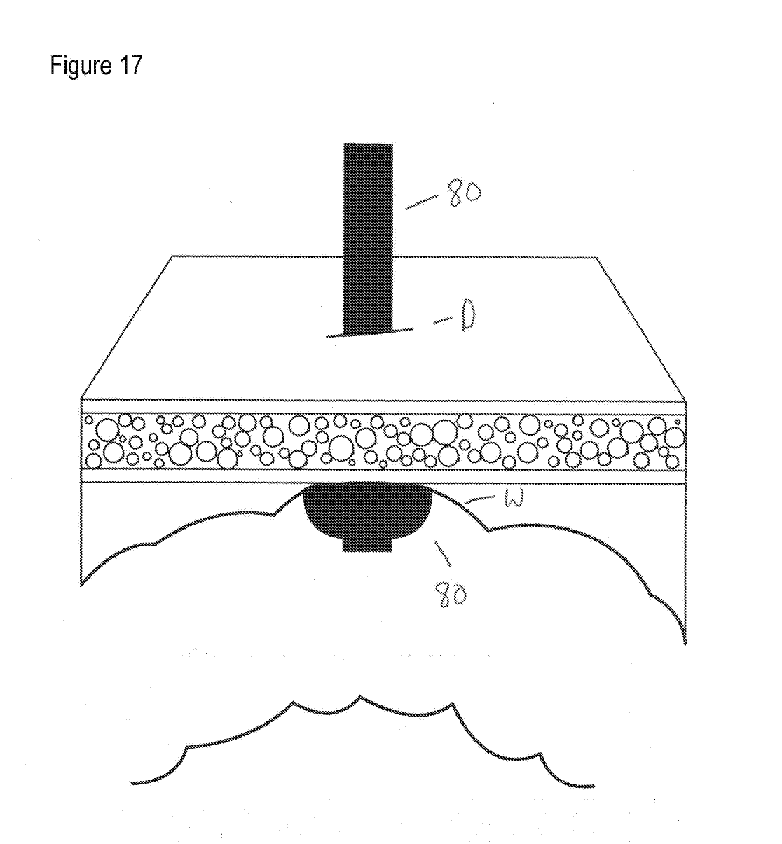

In some embodiments a separate guidewire (FIG. 8) is used to intubate the sheath 20 that has been delivered out of the abdominal wall skin D (FIG. 14). The guidewire 75 has a loop 76 connected at one end thereof, with a non-puncture point 72 at the other end of the guidewire 75. This guidewire 75 may be packaged along with or separately from the packaged device. This guidewire loop 76 emerging at the mouth M (FIGS. 15 and 16) or anus (FIG. 15a) can be linked to a separately packaged gastrointestinal catheter 80 which has a looped end 83 to connect to loop 76 on guidewire. This catheter 80 can then be positioned in the patient by drawing on the end 77 of the wire 75 that is outside the abdominal wall D, thereby drawing the gastrointestinal catheter 80 into position across the visceral wall W and across the abdominal wall D, as illustrated in FIG. 17.

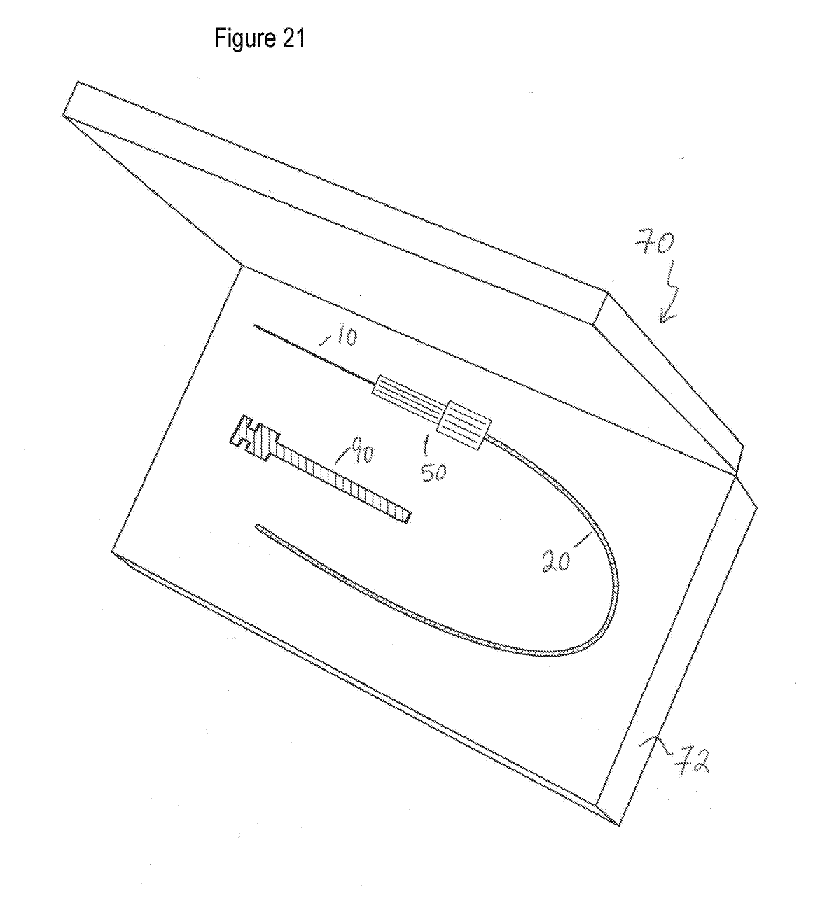

FIG. 21 illustrates one embodiment of a kit containing the system disclosed herein. In this embodiment, kit 70 includes packaging 72 with portions to receive the puncture wire 10/sheath 20 assembly which include a vise lock assembly. The kit 70 of FIG. 21 can also contain a catheter 90 to be advanced over the puncture wire after the puncture wire is secured outside the abdominal wall. The catheter 90 may be of single lumen or coaxial lumen design. The kit 70 of FIG. 21 can also include a sheath locking mechanism such as sheath locking mechanism 60 described above, to enable mounting of the sheath 20 to the endoscope 40, or provided as a separate component within the kit 70 which the user would mount to the sheath 20 after removing it from the packaging (not depicted in FIG. 21). The kit 70 may also include a guidewire such as guidewire 75 with a looped end 76 and a non-sharp second end 72 (not depicted in FIG. 21).

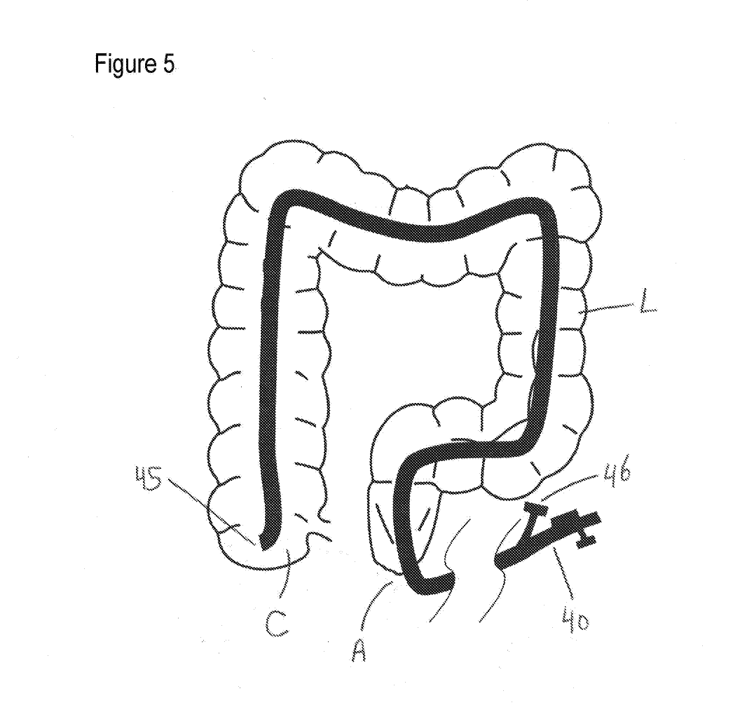

Turning now to the method of use of the system of FIGS. 1-17, FIG. 4 illustrates endoscope 40 inserted through the mouth M and extending up to the stomach S. It is understood that endoscope 40 could similarly be extended further down the gastrointestinal tract, for example to the jejunum J, for puncture and tract creation at that location. FIG. 5 illustrates the method wherein the endoscope 40 inserted through the anus A around the large bowel L, to the cecum C. The endoscope 40 in either method is manipulated under vision so its distal end 45 extends to the site of target puncture. Note the endoscope 40 can be articulated into the location of choice.

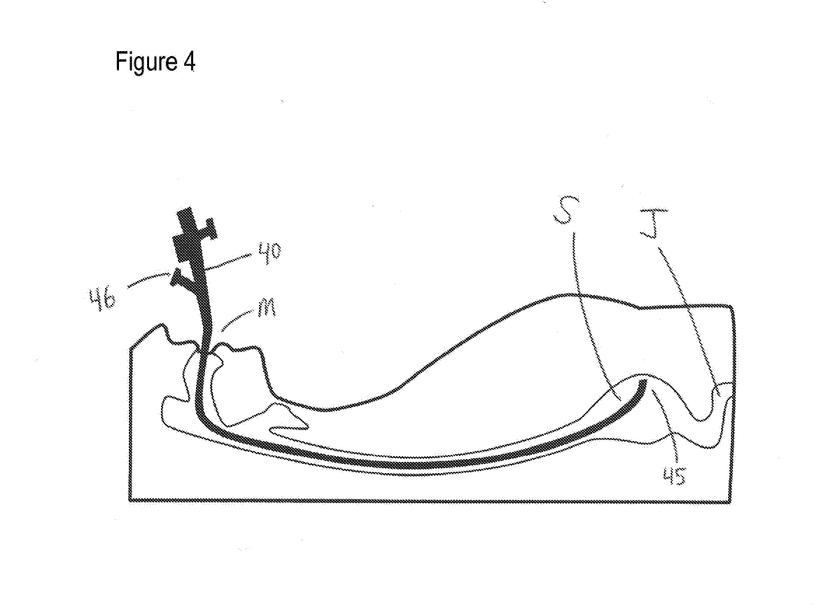

After placement of the endoscope 40 at the desired location, e.g., stomach (FIG. 4), jejunum, or cecum (FIG. 5), the puncture wire 10 and sheath 20, locked together by tightening of the pin vise lock mechanism 50 as described above, are inserted through the working channel 42 of endo scope 40. This may be performed prior to or after introduction of the endoscope 40 into the patient. At this point, the puncture wire tip 21 of puncture wire 10 is retracted and thereby shielded within the protective sheath 20 as depicted in FIG. 1. The puncture wire 10 and sheath 20 are then advanced just distal of the tip 45 of the endoscope 40 (beyond distal opening 47, as shown in FIG. 9), and viewed to make sure they are in the desired anatomical position. Once so positioned, in the embodiment where a Touhy-Borst connecter mounted to the endoscope is utilized, the threaded knob 64 of sheath locking mechanism 60 is rotated in a clockwise fashion to clamp down on the O-ring 68 to reduce its lumen internal diameter, thereby providing a clamping force on the outer wall of sheath 20. This locks the sheath 20 in position, preventing movement with respect to the endoscope 40 and easing advancement of the puncture wire for the surgeon.

To advance the puncture wire 10 further through the scope 40 and sheath 20, actuator 52 of pin vise lock 50 is rotated in a counter-clockwise direction, thereby releasing the locking engagement of the puncture wire 10 and sheath 20. This enables the surgeon to advance the puncture wire 10 through the visceral wall W and skin D as shown in FIG. 9.

In the first embodiment, once the puncture wire 10 is positioned through the skin D (FIG. 9), the sheath 20 is then re-locked in relation to the puncture wire 10 by twisting component 52 of pin vise lock 50 in the clockwise direction 53 (FIG. 10). The two components (puncture wire 10 and sheath 20) are then advanced as a unit further through the working channel 42 of the endoscope 40 and through the visceral wall W and skin D to the position of FIG. 8. Note the sheath 20 has sufficient length 29 (FIG. 10) to permit a length of sheath 29 to be drawn out of the abdominal wall W (FIG. 13) without the pinvise apparatus 50 (FIG. 10) abutting the endoscope port 46 prior to the sheath 20 emerging from the abdominal wall D.

In the next step of this first embodiment, the puncture wire 10 (FIG. 13) is withdrawn from the sheath 20 and from the endoscope. Note that if the sheath 20 and puncture wire 10 are locked together at this point, the pin vise lock 50 needs to be loosened to allow withdrawal of the puncture wire 10 from the sheath 20. Withdrawing the puncture wire 10 from the patient's body leaves the sheath 20 in place extending through the abdominal wall D. The endoscope 40 is kept in position, leaving the sheath 20 in the body, thereby providing a “through and through sheath” as shown in FIG. 14.

A guidewire 75 can then be inserted though the lumen of the sheath 20. The guidewire 75 can be inserted into the sheath 20 in a direction the same as or opposite to the direction of wire puncture through the skin. FIG. 14 shows the guidewire inserted in a direction opposite to wire puncture. Guidewires that can be inserted through the sheath 20 include 0.025 to 0.045 inch guidewires, although other sized guidewires are also contemplated. After insertion of the guidewire 75, the sheath 20 and endoscope 40 are removed from the mouth M or anus A, enabling the wire 75 to obtain “through and through” control of the percutaneous gastrointestinal puncture tract (FIG. 15). Further depicted in FIG. 14 is where the looped end (loop) 76 of the guidewire 75 is advanced through the sheath 20 in an orientation such that the looped end 76 will be positioned at the mouth end of the gastrointestinal tract. In the case of passage through the anus A, large bowel L, and cecum C, the looped end 76 of the guidewire 75 may be advanced in an orientation such that the looped end 76 emerges at the anus A (FIG. 15a). The opposite orientation of loop position (outside the abdominal wall) is contemplated for other applications.

This guidewire loop 76 now emergent at the mouth M (FIG. 15) or anus A (FIG. 15a) can be linked to a separately packaged gastrointestinal catheter 80, shown only at the mouth M but may similarly occur at the anus A. This gastrointestinal catheter 80 bears a looped end 83 to connect to loop 76 on guidewire 75 as shown at the mouth M in FIG. 16. This catheter 80 can then be positioned in the patient by drawing on the end 77 of the wire 75 that is outside the abdominal wall D overlying the stomach S, jejunum J or cecum C, thereby drawing the gastrointestinal catheter 80 into position across the visceral wall W and across the abdominal wall D, as illustrated in FIG. 17.

Thus, as can be appreciated, the protective sheath 20 functions as an “exchange sheath” (or exchange catheter) as after withdrawal of the puncture wire 10 therefrom, it allows for passage of another wire, the guidewire exchange allowing for subsequent function, not limited to engaging a catheter with a looped member to attach to the looped end of guidewire.

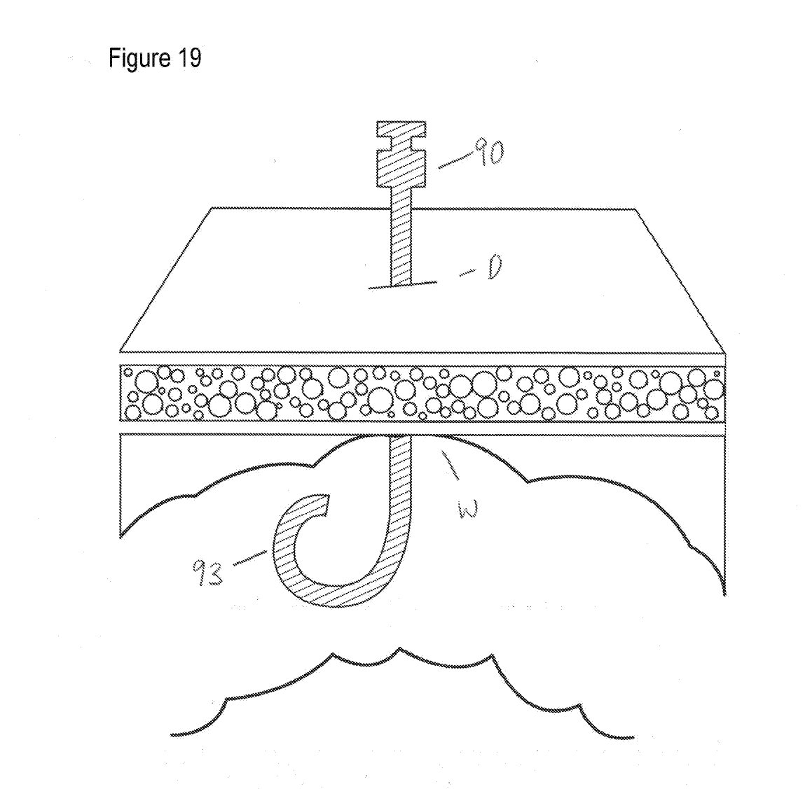

A second embodiment of the system and method of the present invention is illustrated in FIG. 18. In this embodiment, the system and method begins and continues as essentially detailed in the first embodiment above until the puncture wire is emergent at the skin D as in the step of FIG. 9. At this point, a length 27 of wire 10 is drawn further out of the sheath 20 to permit loading of a catheter 90 or other apparatus while still controlling the puncture end 21 of the puncture wire 10. This requires that the length of emergent wire 27 is longer than the catheter 90. At this time the pin vise lock 50 is relocked by rotation in the direction of arrow 53 in (FIG. 10) by twisting in a clockwise direction the actuator 52 of FIG. 3 in the manner described above until the wire 10 is axially fixed in relation to the sheath 20. Catheter 90 or other apparatus is now loaded over the puncture wire 10. With support on the end of the puncture wire 21 with a hemostat clamp to provide a luminal support of the advancement of the catheter 90, the catheter 90 is advanced through the abdominal wall and visceral lumen resulting in the catheter 90 position traversing the abdominal wall D and into the visceral wall W (FIG. 19) in which position the catheter 90 is secured. A loop 93 is shown on the end of the catheter for securing said catheter inside the viscera.

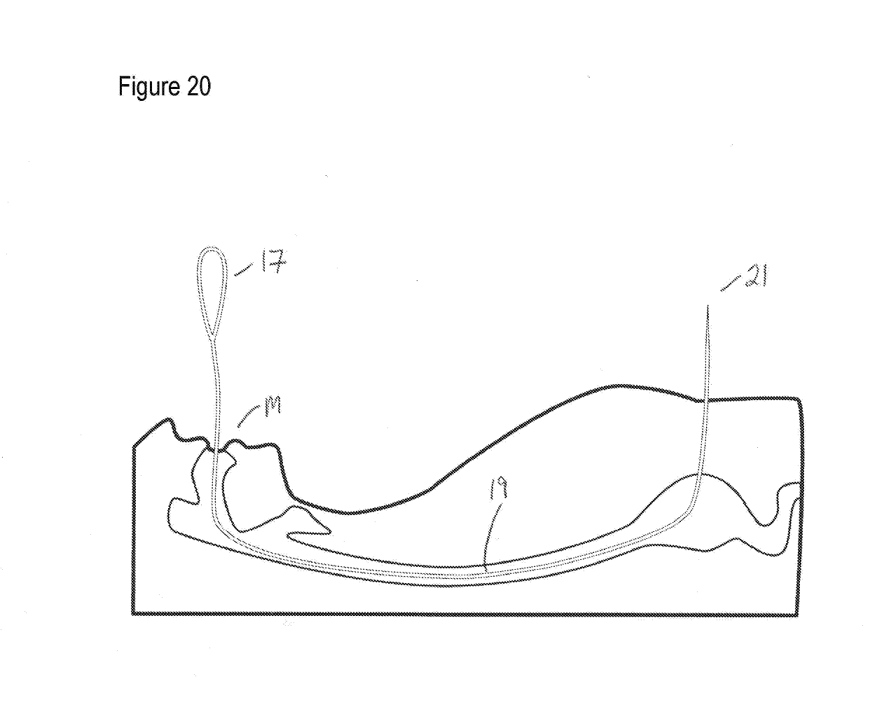

A third embodiment of the system and method of the present invention is illustrated in FIG. 20. The method can utilize the solid construction puncture wire 19 depicted in FIG. 7 with the looped proximal segment 17 and sharp puncture point 21 at the distal end of the wire. In this embodiment, the system and method begins and continues as essentially detailed in the first embodiment above until the puncture wire is emergent at the skin D as in the step of FIG. 9. After the puncture wire is controlled at the skin D, the endoscope 40 and sheath 20 are removed from the mouth M, resulting in position of the puncture wire 19 ‘through and through’ the patient with the loop 17 emergent at the mouth. A gastrointestinal catheter such as catheter 80 of FIG. 16 is connected to the loop 17 on the puncture wire such as via looped member 83 of the catheter 80. The puncture wire point 21 of the puncture wire 19 is then drawn further out the abdominal wall D until the gastrointestinal catheter is in final position as shown in FIG. 17.

It is also contemplated that the characteristics of the puncture wire can be altered. For example, a coating can be applied to improve lubriciousness, and such coating can extend on a portion of or the length of the wire proximal of the tissue puncturing region. Coating with a low friction coefficient material could increase the wire caliber without significantly changing its handling properties.

Also, in some embodiments, portions of the wire can be made thicker, softer or more flexible. For example, the wire can have a thinner portion at the distal portion with a larger diameter at the remaining portion such as the region that contacts the end of the sheath 20 as the sheath is drawn out of the abdomen, or that segment of wire that luminally supports a catheter advanced into the abdomen over the puncture wire.

In some embodiments, the puncture wire can have echogenic properties on part of or its entire length to permit visualization under ultrasound guidance. This may be achieved by exampled with selecting inherently echogenic materials for wire construction (e.g. cobalt/chromium, graphite, teflon, platinum, tungsten, etc.), applying an echogenic coating to the wire, or applying a post-process of chemical abrasion, grit blast (e.g., aluminum oxide, beads, laser etching, chemical treatments, etc.), or other methods to achieve echogenicity.

The protective sheath for the puncture wire may be constructed to be thin walled to permit the entire puncture wire/protective sheath duo to maintain a small enough total diameter for passage through the working channel of the endoscope. Use of materials such as polyimide or PTFE for sheath construction may have beneficial properties for this application. The sheath however can be composed of other materials not limited to extrusion tubing or PTFE.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

What is claimed is:

1. A method for creating a tract in retrograde fashion for percutaneous access to the gastrointestinal tract comprise the steps of:
a) providing a puncture wire having a sharp tissue penetrating tip for penetrating an abdominal wall;
b) providing a sheath with a releasably locking lock mounted on a proximal end, the puncture wire slidable within the sheath and being selectively releasably lockable with the sheath;
c) with the penetrating tip shielded within the sheath and the lock in the locked position to prevent damage to a working channel of an endoscope from an exposed penetrating tip, advancing the puncture wire and sheath together into and through an end of the working channel of the endoscope positioned in a lumen of a target viscera;
d) releasing the lock;
e) advancing the puncture wire in relation to the sheath through the selected wall of the target viscera and the abdominal wall until the puncture wire emerges at a skin of a patient;
f) locking the lock to secure a position of the puncture wire in relation to the sheath;
g) advancing the puncture wire and sheath further out of the abdomen; and
h) removing the puncture wire from the sheath while leaving the sheath in position extending outside the abdominal wall to provide a through sheath to subsequently receive a member therethrough.

2. The method of claim 1, further comprising the step of selecting a target site for puncture under direct visualization prior to advancing the puncture wire through the selected bowel segment.

3. The method of claim 1, wherein the step of advancing the puncture wire includes grasping the lock for support to avoid compression of the sheath onto the wire.

4. The method of claim 1, wherein the puncture wire has a narrower distal segment in relation to a proximal segment, the penetrating tip positioned distal of the narrower distal segment.

5. The method of claim 1, wherein the sheath is one of translucent or transparent.

6. The method of claim 1, further comprising a locking mechanism mountable to the endoscope, the locking mechanism having an opening to receive the sheath and puncture wire therethrough and actuable to secure the sheath to the endoscope.

7. The method of claim 1, wherein the member is a guidewire.

8. The method of claim 7, wherein the guidewire has a loop on one end.

9. The method of claim 8, further comprising the steps of advancing the loop through a mouth of the patient, connecting the loop to a gastrointestinal apparatus and withdrawing the guidewire to pull the gastrointestinal apparatus through the abdominal wall.

10. The method of claim 8, further comprising the steps of advancing the loop through an anus of the patient, connecting the loop to a gastrointestinal apparatus and withdrawing the guidewire to advance the gastrointestinal apparatus through the abdominal wall

11. A method for creating a tract in retrograde fashion for percutaneous access to the gastrointestinal tract comprising the steps of:
a) providing a puncture wire having a sharp tissue penetrating tip for penetrating an abdominal wall;
b) providing a sheath with a releasably locking lock on a proximal end, the puncture wire slidable within the sheath and being selectively releasably lockable with the sheath;
c) with the puncture tip shielded with inside the end of the sheath and the lock in the locked position to prevent damage to a working channel of an endoscope from an exposed penetrating tip, advancing the puncture wire and sheath together into and through an end of a working channel in an endoscope that is positioned in the lumen of the target viscera;
d) releasing the lock;
e) advancing the puncture wire in relation to the sheath, through a selected visceral wall until the puncture wire passes through the abdominal wall and emerges at a skin of a patient.
f) drawing an additional length of the puncture wire out of the skin;
g) locking the lock to secure the puncture wire in position in relation to the sheath;
h) loading a member over the puncture wire at the skin and advancing the member through the abdominal wall and visceral wall into a viscera lumen; and
i) removing the puncture wire and securing the member in position, a portion of the member extending out of the abdominal wall.

12. The method of claim 11, wherein the member is a catheter.

13. The method of claim 11, wherein the lock is a vise lock.

14. The method of claim 11, wherein the puncture wire has a narrower distal segment in relation to a proximal segment, and the penetrating tip is distal to the narrower distal segment.

15. The method of claim 11, wherein prior to advancing the member over the wire into an abdomen, the sheath and puncture wire are releasably fixed to the endoscope.

16. The method of claim 12, wherein the puncture wire is more than about 20 cm longer than the sheath to provide adequate length to load the catheter of sufficient length over the puncture wire outside the abdomen.

17. A method for creating a tract in retrograde fashion for percutaneous access to the gastrointestinal tract comprising the steps of:
a) providing a puncture wire having a sharp tissue penetrating tip at an end for penetrating an abdominal wall;
b) providing a sheath with a releasably locking lock mounted on its proximal end, the puncture wire slidable within the sheath and being selectively releasably lockable with the sheath;
c) with the penetrating tip shielded inside the sheath and the lock in the locked position to prevent movement of the puncture wire and sheath in relation to each other and thereby avoid emergence of the puncture wire tip from the sheath, thus avoiding damage to a working channel of an endoscope from an exposed puncture wire tip, advancing the puncture wire and sheath together into and through the end of the working channel in the endoscope that is positioned in a lumen of a target viscera;
d) releasing the lock:
e) advancing the puncture wire in relation to the sheath, through a selected visceral wall until the puncture wire emerges through the abdominal wall and is controlled at a skin of a patient;
f) removing the endoscope and sheath from the patient in a direction opposite a direction of insertion of the endoscope;
g) connecting a gastrointestinal catheter to the proximal end of the puncture wire located at a natural body orifice;
h) drawing the end of the puncture wire further out of the abdominal wall to draw the gastrointestinal catheter into the target viscera, and
i) removing the puncture wire, leaving the gastrointestinal catheter in proper position.

18. The method of claim 17, wherein the step of drawing the puncture wire further out of the abdominal wall includes further moving an end of the gastrointestinal catheter apparatus out through the abdominal wall.

19. The method of claim 17, wherein the puncture wire has a looped segment at a proximal end opposite the end having the penetrating tip.

20. The method of claim 17, wherein the puncture wire is a solid construction and has a wider proximal segment, a narrower distal segment and a puncture segment narrower than the distal segment containing the penetrating tip.
